(19) United States
(12) Patent Application Publication
(10) Pub. No.: US 2026/0295239 A1
(43) Pub. Date: Oct. 1, 2026

(54) IMPLANTABLE LEADS WITH DIFFERENT AXIAL LENGTH ELECTRODES

(71) Applicant: Medtronic, Inc., Minneapolis, MN (US)

(72) Inventors: Paula Andrea Elma Dassbach, Shoreview, MN (US); Rene A. Molina, Maple Grove, MN (US)

(21) Appl. No.: 19/479,430

(22) PCT Filed: Apr. 3, 2024
(86) PCT No.: PCT/IB2024/053244

§ 371 Date: Oct. 28, 2025

Related U.S. Application Data
(60) Provisional application No. 63/499,099, filed on Apr. 28, 2023.

(51) Int. Cl. A61N 1/05

(52) U.S. Cl. CPC A61N 1/05

(57) ABSTRACT

Devices, systems, and techniques are disclosed for delivering electrical stimulation therapy and/or sensing of physiological signals from a variety of different positions on one or more medical leads. For example, a medical lead may include a cylindrical lead body defining a longitudinal axis, a proximal portion, and a distal portion, and a first electrode comprising a first length parallel with the longitudinal axis, the first electrode disposed at a first circumferential position around the cylindrical lead body. The medical lead may include a plurality of second electrodes, each electrode of the plurality of second electrodes including one or more second lengths parallel with the longitudinal axis and different than the first length, and where the plurality of second electrodes disposed at a second circumferential position of the cylindrical lead body different than the first circumferential position. Each of the plurality of second electrodes may be disposed at different longitudinal positions.

This application claims priority from U.S. Provisional Patent Application 63/499,099, filed 28 Apr. 2023, the entire content of which is incorporated herein by reference.

### TECHNICAL FIELD

This disclosure generally relates to electrical stimulation and recording.

### BACKGROUND

Medical devices may be external or implanted, and may be used to deliver electrical stimulation therapy to various tissue sites of a patient to treat a variety of symptoms or conditions such as chronic pain, tremor, Parkinson's disease, other movement disorders, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Hence, electrical stimulation may be used in different therapeutic applications, such as deep brain stimulation (DBS), spinal cord stimulation (SCS), pelvic stimulation, gastric stimulation, or peripheral nerve field stimulation (PNFS).

A clinician may select values for a number of programmable parameters in order to define the electrical stimulation therapy to be delivered by the implantable stimulator to a patient. For example, the clinician may select one or more electrodes for delivery of the stimulation, a polarity of each selected electrode, a voltage or current amplitude, a pulse width, and a pulse frequency as stimulation parameters. A set of parameters, such as a set including electrode combination, electrode polarity, voltage or current amplitude, pulse width and pulse rate, may be referred to as a program in the sense that they define the electrical stimulation therapy to be delivered to the patient.

### SUMMARY

In general, the disclosure is directed to devices, systems, and techniques for delivering electrical signals from or sensing electrical signals by different locations of one or more medical leads. A medical lead may carry electrodes at different positions around a perimeter of the lead, and different electrodes may have different longitudinal, or axial, lengths along the length of the lead. In this manner, different electrode combinations may be selected from the available variety of electrodes on the lead to achieve desired electrical field distributions and/or sensing vectors that may not be otherwise achievable.

The lead may have electrodes disposed at different circumferential positions around the lead, such as at the distal end of the lead. At one circumferential position, the lead may carry a single electrode that is substantially longer in the direction of the axis of the lead than the electrode width around the circumference. At a different circumferential position, the lead may carry multiple electrodes that are each shorter than the single electrode. For example, the multiple electrodes may occupy the same longitudinal length on the lead than the single electrode at the different circumferential position. The medical lead may have three, four, five, or more circumferential positions, or columns, that have one, two, three, four, five or more electrodes at each circumferential position. Each column may include the same number of electrodes or different number of electrodes. These different electrode patterns may enable the medical lead to provide a variety of different stimulation and/or sensing electrode configurations.

In one example, a medical lead includes a cylindrical lead body defining a longitudinal axis, a proximal portion, and a distal portion; a first electrode comprising a first length parallel with the longitudinal axis, the first electrode disposed at a first circumferential position around the cylindrical lead body; and a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions.

In another example, a system includes a medical lead comprising: a cylindrical lead body defining a longitudinal axis, a proximal portion, and a distal portion; a first electrode comprising a first length parallel with the longitudinal axis, the first electrode disposed at a first circumferential position around the cylindrical lead body; a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions; a plurality of contacts disposed on the proximal portion of the cylindrical lead; and a plurality of conductors electrically coupling the first electrode and the plurality of second electrodes to respective contacts of the plurality of contacts; and an implantable medical device configured to: electrically couple to the plurality of contacts of the medical lead; and deliver electrical stimulation via at least two electrodes of the first electrode and the plurality of second electrodes of the medical lead.

In another example, a medical lead includes a cylindrical lead body defining a longitudinal axis, a proximal portion, and a distal portion; a first electrode comprising a first length parallel with the longitudinal axis, the first electrode disposed at a first circumferential position around the cylindrical lead body; a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions; a third electrode at a third circumferential position different than the first circumferential position and the second circumferential position, wherein the third electrode has a third length equal to the first length; and a plurality of fourth electrodes at a fourth circumferential position different than the first circumferential position and the second circumferential position, wherein the first and third circumferential positions are on opposing sides of the cylindrical lead body, and wherein the second and fourth circumferential positions are on opposing sides of the cylindrical lead body.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1A is a conceptual diagram illustrating an example system that includes an implantable medical device (IMD) configured to deliver DBS to a patient using a medical lead according to an example of the techniques of the disclosure.

FIG. 1B is an x-ray image of an example patient with a medical lead implanted from a posterior entry position into the cranium.

FIG. 2 is a block diagram of the example IMD of FIG. 1 for delivering DBS therapy according to an example of the techniques of the disclosure.

FIG. 3 is a block diagram of the external programmer of FIG. 1 for controlling delivery of DBS therapy according to an example of the techniques of the disclosure.

FIGS. 4A and 4B are conceptual diagrams of example leads with respective electrodes carried by the lead.

FIGS. 5A, 5B, 5C, and 5D are conceptual diagrams of example cross-sectional views of electrodes disposed around a perimeter of a lead at a particular longitudinal location.

FIG. 6 is a perspective view of an example lead with longitudinally orientated electrodes at respective circumferential positions.

FIG. 7 is a perspective view of an example lead with longitudinally orientated electrodes and longitudinal segmented electrodes at respective circumferential positions.

FIG. 8 is a perspective view of an example lead with different longitudinal segmented electrodes at respective circumferential positions.

FIG. 9 is a perspective view of an example lead with longitudinally orientated electrodes and longitudinal segmented electrodes at respective circumferential positions.

FIG. 10 is a perspective view of an example lead with longitudinally orientated electrodes and smaller longitudinal segmented electrodes at a distal end of the lead at respective circumferential positions.

FIG. 11 is a perspective view of an example lead with longitudinally orientated electrodes and smaller longitudinal segmented electrodes disposed proximal from the end of the lead and at respective circumferential positions.

FIG. 12 is a perspective view of an example lead with longitudinally orientated electrodes and smaller longitudinal segmented electrodes disposed between the longer longitudinally orientated electrodes.

FIG. 13 is a perspective view of an example lead with longitudinally orientated electrodes and smaller longitudinal segmented electrodes disposed proximal from the end of the lead at one circumferential position and smaller longitudinal segmented electrodes disposed distal from the end of the lead at a different circumferential position.

FIG. 14 is a perspective view of an example lead with different sized segmented electrodes at different longitudinal and circumferential positions.

FIGS. 15-17 are perspective views of an example leads with longitudinally orientated electrodes and different sized segmented electrodes at different longitudinal and circumferential positions.

FIG. 18 is a flowchart illustrating an example technique for identifying electrode orientation and selecting electrodes from a lead with electrodes at different circumferential positions around a perimeter of the lead.

### DETAILED DESCRIPTION

This disclosure describes various devices and system that include medical leads having electrodes disposed at different positions around a perimeter of the lead. The lead can then be used to sense electrical signals from and/or deliver electrical stimulation to target tissue locations. A patient may suffer from one or more symptoms treatable by electrical stimulation therapy. For example, a patient may suffer from brain disorder such as Parkinson's disease, Alzheimer's disease, or another type of movement disorder. Deep brain stimulation (DBS) may be an effective treatment to reduce the symptoms associated with such disorders. However, efficacy of stimulation therapy may be reliant on providing appropriate electrical fields to a target region of tissue and/or sensing electrical signals using sensing vectors appropriate to detect one or more physiological signals. Stimulation of tissue outside of the target region may elicit undesirable effects, reduce the efficacy of the therapy, or require more energy than may be necessary for treatment.

As described herein, various leads are described that have different electrode positions around a perimeter of the lead and/or at different axial, or longitudinal, positions along the lead. In general, the leads described herein have one or more electrodes that have a longitudinal dimension (e.g., the dimension parallel with the central axis of the lead) greater than a circumferential dimension (e.g., the length of the electrode around the perimeter of the lead that may be orthogonal to the axis of the lead). In some examples, these electrodes with longer longitudinal dimension than circumferential dimension may be referred to as “vertical electrodes” because they are oriented to have more material vertically, or along the longitudinal direction, than around the perimeter of the lead. In some examples, all electrodes on the lead may be vertical electrodes, but in other examples, some electrodes may be vertical electrodes and one or more other electrodes on the lead may have a width, or circumferential dimension, greater than the longitudinal dimension.

A lead may have vertical electrodes at different positions around the perimeter of the lead, such as different vertical electrodes at 2, 3, 4, or more different positions around the perimeter of the lead. These electrodes may be equally spaced around the perimeter of the lead or unevenly spaced around the perimeter of the lead (different widths between each electrode and/or different circumferential dimensions for one or more of the electrodes). In some examples, electrodes at different circumferential positions of the lead may have the same longitudinal dimensions or have different longitudinal dimensions.

In any of the leads described herein, the electrodes disposed at different circumferential positions may have equal circumferential dimensions, or widths, around the circumference or perimeter of the lead. In other examples, electrodes at different circumferential positions may have one or more different circumferential dimensions, or widths, around the circumference or perimeter of the lead. In some examples, electrodes with larger longitudinal lengths may have narrower circumferential widths than electrodes with smaller longitudinal lengths. This variation in electrode width may enable electrodes with longer longitudinal lengths to have surface areas closer to that of electrodes with shorter longitudinal lengths if the widths were the same. In some examples, the widths of each electrode may be varied according to their length such that the surface area of each electrode are substantially similar (e.g., equal or within a small tolerance such as 5% deviation of each other). These similar surface areas of the electrodes may enable the electrodes to have similar impedances to each other to facilitate sensing and/or stimulation.

The leads described herein having electrodes with longitudinally long electrodes at different positions around the perimeter of the lead may enable an implantable medical device (IMD) to deliver electrical stimulation to certain target tissue while avoiding stimulation to other tissue areas. In this manner, one or more electrodes on the lead may be used in a custom electrode combination to provide desired electrical field shapes for targeting the target tissue. In one example, a longer electrode may generate an electric field for a target tissue that is stretched along the longitudinal length of the lead. In addition, or alternatively, the IMD may utilize different electrode combinations of electrodes on one or more leads to provide different sensing vectors. For example, a shorter electrode may be selected nearer a target tissue area and used with a longer electrode at a different location of the lead in order to focus sensing at the target tissue area.

Although this disclosure is directed to DBS therapy, the systems, devices, and techniques described herein may similarly detect movement of leads and electrodes implanted outside of the brain, such as near other nerves or muscles for different diagnostic or therapeutic applications, such as spinal cord stimulation (SCS), pelvic floor stimulation, gastric stimulation, or peripheral nerve field stimulation (PNFS). Moreover, a human patient is described for example purposes herein, but similar systems, devices, and techniques may be used for other animals in other examples.

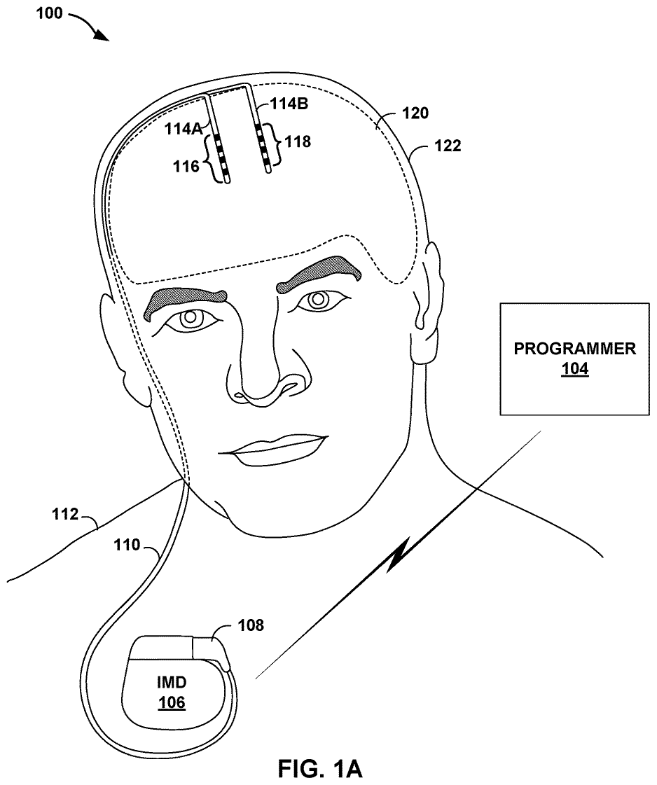

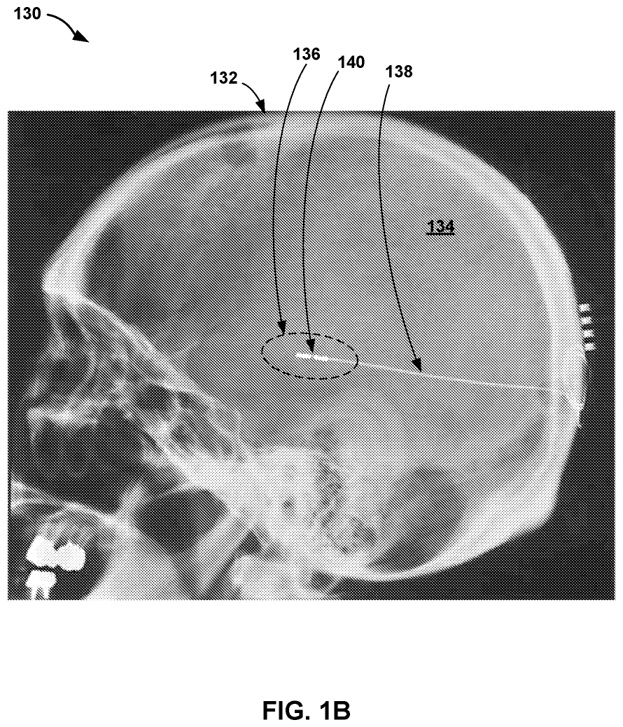

FIG. 1A is a conceptual diagram illustrating an example system 100 that includes an implantable medical device (IMD) 106 configured to deliver DBS to patient 122 via electrodes of any of leads according to an example of the techniques of the disclosure. As shown in the example of FIG. 1A, example system 100 includes medical device programmer 104, implantable medical device (IMD) 106, lead extension 110, and leads 114A and 114B with respective sets of electrodes 116, 118. In the example shown in FIG. 1A, electrodes 116, 118 of leads 114A, 114B are positioned to deliver electrical stimulation to a tissue site within brain 120, such as a deep brain site under the dura mater of brain 120 of patient 112. In some examples, delivery of stimulation to one or more regions of brain 120, such as the subthalamic nucleus, globus pallidus or thalamus, may be an effective treatment to manage movement disorders, such as Parkinson's disease. Some or all of electrodes 116, 118 also may be positioned to sense neurological brain signals within brain 120 of patient 112. In some examples, some of electrodes 116, 118 may be configured to sense neurological brain signals and others of electrodes 116, 118 may be configured to deliver adaptive electrical stimulation to brain 120. In other examples, all of electrodes 116, 118 are configured to both sense neurological brain signals and deliver adaptive electrical stimulation to brain 120. As described herein, one, some, or all of electrodes 116, 118 may be vertical electrodes that have a larger longitudinal dimension than the circumferential dimension orthogonal to the axis the respective lead.

IMD 106 includes a therapy module (e.g., which may include processing circuitry, signal generation circuitry or other electrical circuitry configured to perform the functions attributed to IMD 106) that includes a stimulation generator configured to generate and deliver electrical stimulation therapy to patient 112 via a subset of electrodes 116, 118 of leads 114A and 114B, respectively. The subset of electrodes 116, 118 that are used to deliver electrical stimulation to patient 112, and, in some cases, the polarity of the subset of electrodes 116, 118, may be referred to as a stimulation electrode combination. As described in further detail below, the stimulation electrode combination can be selected for a particular patient 112 and target tissue site (e.g., selected based on the patient condition). The group of electrodes 116, 118 includes at least one electrode and can include a plurality of electrodes. The plurality of electrodes 116 and/or 118 generally have two or more electrodes of the lead located at different positions around the perimeter, or circumference, of the respective lead (e.g., different positions around a longitudinal axis of the lead). In some examples, the plurality of electrodes 116 and/or 118 may also have two or more electrodes at different longitudinal positions along the length of the respective lead. At least one of these electrodes may be vertical electrodes that have a dimension in the longitudinal direction that is longer than the circumferential direction of the electrode is wide. In this manner, these vertical electrodes may provide a lengthened electric field compared with electrodes that generally are wider than longer.

In some examples, the neurological signals (e.g., an example type of electrical signals) sensed within brain 120 may reflect changes in electrical current produced by the sum of electrical potential differences across brain tissue. Examples of neurological brain signals include, but are not limited to, electrical signals generated from local field potentials (LFP) sensed within one or more regions of brain 120, such as an electroencephalogram (EEG) signal, or an electrocorticogram (ECoG) signal. Local field potentials, however, may include a broader genus of electrical signals within brain 120 of patient 112.

In some examples, the neurological brain signals that are used to select a stimulation electrode combination may be sensed within the same region of brain 120 as the target tissue site for the electrical stimulation. As previously indicated, these tissue sites may include tissue sites within anatomical structures such as the thalamus, subthalamic nucleus or globus pallidus of brain 120, as well as other target tissue sites. The specific target tissue sites and/or regions within brain 120 may be selected based on the patient condition. Thus, due to these differences in target locations, in some examples, the electrodes used for delivering electrical stimulation may be different than the electrodes used for sensing neurological brain signals. In other examples, the same electrodes may be used to deliver electrical stimulation and sense brain signals. However, this configuration would require the system to switch between stimulation generation and sensing circuitry and may reduce the time the system can sense brain signals.

Electrical stimulation generated by IMD 106 may be configured to manage a variety of disorders and conditions. In some examples, the stimulation generator of IMD 106 is configured to generate and deliver electrical stimulation pulses to patient 112 via electrodes of a selected stimulation electrode combination. However, in other examples, the stimulation generator of IMD 106 may be configured to generate and deliver a continuous wave signal, e.g., a sine wave or triangle wave. In either case, a stimulation generator within IMD 106 may generate the electrical stimulation therapy for DBS according to a therapy program that is selected at that given time in therapy. In examples in which IMD 106 delivers electrical stimulation in the form of stimulation pulses, a therapy program may include a set of therapy parameter values (e.g., stimulation parameters), such as a stimulation electrode combination for delivering stimulation to patient 112, pulse frequency, pulse width, and a current or voltage amplitude of the pulses. As previously indicated, the electrode combination may indicate the specific electrodes 116, 118 that are selected to deliver stimulation signals to tissue of patient 112 and the respective polarities of the selected electrodes. IMD 106 may deliver electrical stimulation intended to contribute to a therapeutic effect. In some examples, IMD 106 may also, or alternatively, deliver electrical stimulation intended to be sensed by other electrode and/or elicit a physiological response, such as an evoked compound action potential (ECAP), that can be sensed by electrodes.

IMD 106 may be implanted within a subcutaneous pocket below the clavicle, or, alternatively, on or within cranium 122 or at any other suitable site within patient 112. Generally, IMD 106 is constructed of a biocompatible material that resists corrosion and degradation from bodily fluids. IMD 106 may comprise a hermetic housing to substantially enclose components, such as a processor, therapy module, and memory.

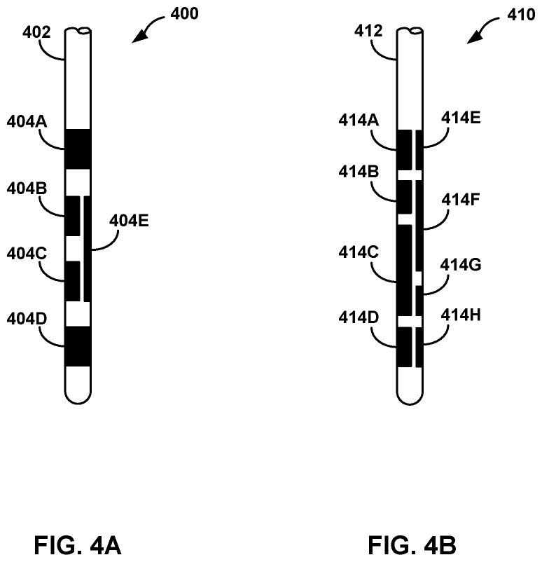

As shown in FIG. 1A, implanted lead extension 110 is coupled to IMD 106 via connector 108 (also referred to as a connector block or a header of IMD 106). In the example of FIG. 1A, lead extension 110 traverses from the implant site of IMD 106 and along the neck of patient 112 to cranium 122 of patient 112 to access brain 120. In the example shown in FIG. 1A, leads 114A and 114B (collectively “leads 114”) are implanted within the right and left hemispheres, respectively, of patient 112 in order deliver electrical stimulation to one or more regions of brain 120, which may be selected based on the patient condition or disorder controlled by therapy system 100. The specific target tissue site and the stimulation electrodes used to deliver stimulation to the target tissue site, however, may be selected, e.g., according to the identified patient behaviors and/or other sensed patient parameters. Other lead 114 and IMD 106 implant sites are contemplated. For example, IMD 106 may be implanted on or within cranium 122, in some examples. Or leads 114 may be implanted within the same hemisphere or IMD 106 may be coupled to a single lead implanted in a single hemisphere. Although leads 114 may have ring electrodes at different longitudinal positions as shown in FIG. 1A, leads 114 may have electrodes disposed at different positions around the perimeter of the lead (e.g., different circumferential positions for a cylindrical shaped lead) as shown in the examples of FIGS. 4A and 4B.

Leads 114 illustrate an example lead set that include axial leads carrying ring electrodes disposed at different axial positions and, in some examples, different longitudinal positions. As described herein, lead array geometries may be used in which electrodes are disposed at different respective longitudinal positions and different positions around the perimeter of the lead, while one or more of the electrodes are longer in the longitudinal direction than wide around the perimeter of the lead.

Although leads 114 are shown in FIG. 1A as being coupled to a common lead extension 110, in other examples, leads 114 may be coupled to IMD 106 via separate lead extensions or directly to connector 108. Leads 114 may be positioned to deliver electrical stimulation to one or more target tissue sites within brain 120 to manage patient symptoms associated with a movement disorder of patient 112. Leads 114 may be implanted to position electrodes 116, 118 at desired locations of brain 120 through respective holes in cranium 122. Leads 114 may be placed at any location within brain 120 such that electrodes 116, 118 are capable of providing electrical stimulation to target tissue sites within brain 120 during treatment. For example, electrodes 116, 118 may be surgically implanted under the dura mater of brain 120 or within the cerebral cortex of brain 120 via a burr hole in cranium 122 of patient 112, and electrically coupled to IMD 106 via one or more leads 114.

In the example shown in FIG. 1A, electrodes 116, 118 of leads 114 are shown as ring electrodes, but at least one or more electrodes are only disposed partially around the perimeter of the respective lead. In this manner, one or more ring electrodes may be used on the same lead as one or more electrodes that only wrap partially around the perimeter of the respective lead. Ring electrodes may be used in DBS applications because they are relatively simple to program and are capable of delivering an electrical field to any tissue adjacent to electrodes 116, 118. Ring electrodes may also provide a larger surface area as a return electrode. In other examples, electrodes 116, 118 may have different configurations. For example, in some examples, at least some of the electrodes 116, 118 of leads 114 may have an electrode array geometry that is capable of producing shaped electrical fields with one or more electrodes that are only wrapped partially around the perimeter of the lead and extend lengthwise along the lead in the longitudinal direction longer than the width of the electrode wrapped around the perimeter. The electrode array geometry may include multiple electrodes (e.g., partial ring or segmented electrodes) around the outer perimeter of each lead 114, rather than one ring electrode, such as shown in FIGS. 4A and 4B. In this manner, electrical stimulation may be directed in a specific direction from leads 114 to enhance therapy efficacy and reduce possible adverse side effects from stimulating a large volume of tissue. In some examples, a housing of IMD 106 may include one or more stimulation and/or sensing electrodes. In alternative examples, leads 114 may have shapes other than elongated cylinders as shown in FIG. 1A. For example, leads 114 may be paddle leads, spherical leads, bendable leads, or any other type of shape effective in treating patient 112 and/or minimizing invasiveness of leads 114.

In the example shown in FIG. 1A, IMD 106 includes a memory to store a plurality of therapy programs that each define a set of therapy parameter values. In some examples, IMD 106 may select a therapy program from the memory based on various parameters, such as sensed patient parameters and the identified patient behaviors. IMD 106 may generate electrical stimulation based on the selected therapy program to manage the patient symptoms associated with a movement disorder.

External programmer 104 wirelessly communicates with IMD 106 as needed to provide or retrieve therapy information. Programmer 104 is an external computing device that the user, e.g., a clinician and/or patient 112, may use to communicate with IMD 106. For example, programmer 104 may be a clinician programmer that the clinician uses to communicate with IMD 106 and program one or more therapy programs for IMD 106. Alternatively, programmer 104 may be a patient programmer that allows patient 112 to select programs and/or view and modify therapy parameters. The clinician programmer may include more programming features than the patient programmer. In other words, more complex or sensitive tasks may only be allowed by the clinician programmer to prevent an untrained patient from making undesirable changes to IMD 106. IMD 106 may also transmit notifications to programmer 104 for delivery to a user in response to detecting that one of leads 114 has moved with respect to tissue. Programmer 104 may enter a new programming session for the user to select new stimulation parameters for subsequent therapy.

When programmer 104 is configured for use by the clinician, programmer 104 may be used to transmit initial programming information to IMD 106. This initial information may include hardware information, such as the type of leads 114 and the electrode arrangement, the position of leads 114 within brain 120, the configuration of electrode array 116, 118, initial programs defining therapy parameter values, and any other information the clinician desires to program into IMD 106. Programmer 104 may also be capable of completing functional tests (e.g., measuring the impedance of electrodes 116, 118 of leads 114). In some examples, programmer 104 may receive sensed signals or representative information and perform the same techniques and functions attributed to IMD 106 herein. In other examples, a remote server (e.g., a standalone server or part of a cloud service) may perform the functions attributed to IMD 106, programmer 104, or any other devices described herein.

The clinician may also store therapy programs within IMD 106 with the aid of programmer 104. During a programming session, the clinician may determine one or more therapy programs that may provide efficacious therapy to patient 112 to address symptoms associated with the patient condition, and, in some cases, specific to one or more different patient states, such as a sleep state, movement state or rest state. For example, the clinician may select one or more stimulation electrode combination with which stimulation is delivered to brain 120. During the programming session, the clinician may evaluate the efficacy of the specific program being evaluated based on feedback provided by patient 112 or based on one or more physiological parameters of patient 112 (e.g., muscle activity, muscle tone, rigidity, tremor, etc.). Alternatively, identified patient behavior from video information may be used as feedback during the initial and subsequent programming sessions. Programmer 104 may assist the clinician in the creation/identification of therapy programs by providing a methodical system for identifying potentially beneficial therapy parameter values.

Programmer 104 may also be configured for use by patient 112. When configured as a patient programmer, programmer 104 may have limited functionality (compared to a clinician programmer) in order to prevent patient 112 from altering critical functions of IMD 106 or applications that may be detrimental to patient 112. In this manner, programmer 104 may only allow patient 112 to adjust values for certain therapy parameters or set an available range of values for a particular therapy parameter.

Programmer 104 may also provide an indication to patient 112 when therapy is being delivered, when patient input has triggered a change in therapy or when the power source within programmer 104 or IMD 106 needs to be replaced or recharged. For example, programmer 112 may include an alert LED, may flash a message to patient 112 via a programmer display, generate an audible sound or somatosensory cue to confirm patient input was received, e.g., to indicate a patient state or to manually modify a therapy parameter.

System 100 may be implemented to provide chronic stimulation therapy to patient 112 over the course of several months or years. However, system 100 may also be employed on a trial basis to evaluate therapy before committing to full implantation. If implemented temporarily, some components of system 100 may not be implanted within patient 112. For example, patient 112 may be fitted with an external medical device, such as a trial stimulator, rather than IMD 106. The external medical device may be coupled to percutaneous leads or to implanted leads via a percutaneous extension. If the trial stimulator indicates DBS system 100 provides effective treatment to patient 112, the clinician may implant a chronic stimulator within patient 112 for relatively long-term treatment.

Although IMD 106 is described as delivering electrical stimulation therapy to brain 120, IMD 106 may be configured to direct electrical stimulation to other anatomical regions of patient 112 in other examples. In other examples, system 100 may include an implantable drug pump in addition to, or in place of, IMD 106. Further, an IMD may provide other electrical stimulation such as spinal cord stimulation to treat a movement disorder.

The architecture of system 100 illustrated in FIG. 1A is shown as an example. The techniques and devices as set forth in this disclosure may be implemented in the example system 100 of FIG. 1A, as well as other types of systems not described specifically herein. Nothing in this disclosure should be construed so as to limit the techniques of this disclosure to the example architecture illustrated by FIG. 1A.

FIG. 1B is an x-ray image of an example patient with a medical lead 138 implanted from a posterior entry position into the cranium 132 and to a target brain tissue 136. System 130 may include lead 138 coupled to an IMD (not shown). System 130 may be similar to system 100 of FIG. 100. As shown in FIG. 1B, lead 138 may be similar to any of leads 114 or other leads herein and may travel through brain 134 in order for electrodes 140 to be disposed near and/or within target brain tissue 136. Electrodes 140 may be similar to any of electrodes 116, 118 or other electrodes described herein. Since one or more of the electrodes 140 may be vertical electrodes with a longer length along the longitudinal axis of lead 138 than the width of the electrodes around the perimeter of lead 138. These electrodes 140 may be suited to generate an electrical field that has a longer length which may be appropriate for treating target brain tissue 136 that also has a longer dimension generally orientated along the length of electrodes 140.

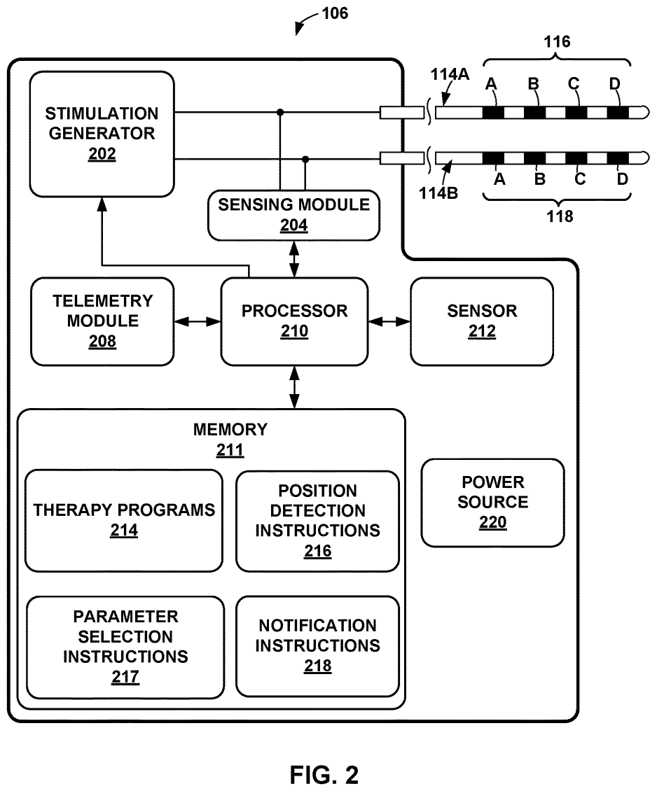

FIG. 2 is a block diagram of the example IMD 106 of FIG. 1 for delivering DBS therapy and/or sensing signals from the patient. In the example shown in FIG. 2, IMD 106 includes processor 210, memory 211, stimulation generator 202, sensing module 204, switch module 206, telemetry module 208, sensor 212, and power source 220. Each of these modules may be or include electrical circuitry configured to perform the functions attributed to each respective module. For example, processor 210 may include processing circuitry, switch module 206 may include switch circuitry, sensing module 204 may include sensing circuitry, and telemetry module 208 may include telemetry circuitry. Switch module 204 may not be necessary for multiple current source and sink configurations. Memory 211 may include any volatile or non-volatile media, such as a random-access memory (RAM), read only memory (ROM), non-volatile RAM (NVRAM), electrically erasable programmable ROM (EEPROM), flash memory, and the like. Memory 211 may store computer-readable instructions that, when executed by processor 210, cause IMD 106 to perform various functions. Memory 211 may be a storage device or other non-transitory medium.

In the example shown in FIG. 2, memory 211 stores therapy programs 214 that include respective stimulation parameter sets that define therapy. Each stored therapy program 214 defines a particular set of electrical stimulation parameters (e.g., a therapy parameter set), such as a stimulation electrode combination, electrode polarity, current or voltage amplitude, pulse width, and pulse rate. In some examples, individual therapy programs may be stored as a therapy group, which defines a set of therapy programs with which stimulation may be generated. The stimulation signals defined by the therapy programs of the therapy group may be delivered together on an overlapping or non-overlapping (e.g., time-interleaved) basis.

Memory 211 may also include position detection instructions 216 that define the orientation of each electrodes 116, 118 with respect to the anatomy of the patient. This orientation of the electrodes may be used in the selection of a subset of electrodes for stimulation and/or sensing. Memory 211 may also include parameter selection instructions 217 and notification instructions 218. Parameter selection instructions 217 may include instructions that control processor 210 selecting different stimulation parameter values such as electrode combinations, amplitudes, pulse frequencies, or other parameter values that define stimulation and/or sensing from the electrodes of the lead. Notification instructions 218 may define instructions that control processor 210 actions such as transmitting an alert or other notification to an external device, such as programmer 104, that indicates any issues related to stimulation, sensing, or aspects or device status.

In some examples, the sense and stimulation electrode combinations may include the same subset of electrodes 116, 118, a housing of IMD 106 functioning as an electrode, or may include different subsets or combinations of such electrodes. Thus, memory 211 can store a plurality of sense electrode combinations and, for each sense electrode combination, store information identifying the stimulation electrode combination that is associated with the respective sense electrode combination. The associations between sense and stimulation electrode combinations can be determined, e.g., by a clinician or automatically by processor 210. In some examples, corresponding sense and stimulation electrode combinations may comprise some or all of the same electrodes. In other examples, however, some or all of the electrodes in corresponding sense and stimulation electrode combinations may be different. For example, a stimulation electrode combination may include more electrodes than the corresponding sense electrode combination in order to increase the efficacy of the stimulation therapy. In some examples, as discussed above, stimulation may be delivered via a stimulation electrode combination to a tissue site that is different than the tissue site closest to the corresponding sense electrode combination but is within the same region, e.g., the thalamus, of brain 120 in order to mitigate any irregular oscillations or other irregular brain activity within the tissue site associated with the sense electrode combination. In other examples, the electrodes that deliver stimulation may be carried by a lead implanted in a different region of the brain than a different lead that carries the sensing electrodes.

Stimulation generator 202, under the control of processor 210, generates stimulation signals for delivery to patient 112 via selected combinations of electrodes 116, 118. An example range of electrical stimulation parameters believed to be effective in DBS to manage a movement disorder of patient include:

1. Pulse Rate, i.e., Frequency: between approximately 0.1 Hertz and approximately 500 Hertz, such as between approximately 0.1 to 10 Hertz, approximately 40 to 185 Hertz, or such as approximately 140 Hertz.

2. In the case of a voltage controlled system, Voltage Amplitude: between approximately 0.1 volts and approximately 50 volts, such as between approximately 2 volts and approximately 3 volts.

3. In the alternative case of a current controlled system, Current Amplitude: between approximately 0.2 milliamps to approximately 100 milliamps, such as between approximately 1.3 milliamps and approximately 2.0 milliamps.

4. Pulse Width: between approximately 10 microseconds and approximately 5000 microseconds, such as between approximately 100 microseconds and approximately 1000 microseconds, or between approximately 180 microseconds and approximately 450 microseconds.

Accordingly, in some examples, stimulation generator 202 generates electrical stimulation signals in accordance with the electrical stimulation parameters noted above. Other ranges of therapy parameter values may also be useful, and may depend on the target stimulation site within patient 112. While stimulation pulses are described, stimulation signals may be of any form, such as continuous-time signals (e.g., sine waves) or the like. Stimulation signals configured to elicit ECAPs or other evoked physiological signals may be similar or different from the above parameter value ranges.

Processor 210 may include fixed function processing circuitry and/or programmable processing circuitry, and may comprise, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processor 210 herein may be embodied as firmware, hardware, software or any combination thereof. Processor 210 may control stimulation generator 202 according to therapy programs 214 stored in memory 211 to apply particular stimulation parameter values specified by one or more of programs, such as voltage amplitude or current amplitude, pulse width, or pulse rate.

In the example shown in FIG. 2, the set of electrodes 116 includes electrodes 116A, 116B, 116C, and 116D, and the set of electrodes 118 includes electrodes 118A, 118B, 118C, and 118D. In some examples, processor 210 may control a switch module to apply the stimulation signals generated by stimulation generator 202 to selected combinations of electrodes 116, 118. In some examples, stimulation generator 202 may comprise multiple voltage or current sources and sinks that are coupled to respective electrodes to drive the electrodes as cathodes or anodes. In this example, IMD 106 may include switches or other components that selectively connect or disconnect sensing module 204 from the lines between stimulation generator 202 and respective electrodes 116, 118.

In other examples, a switch module may couple stimulation signals to selected conductors within leads 114, which, in turn, deliver the stimulation signals across selected electrodes 116, 118. The switch module may be a switch array, switch matrix, multiplexer, or any other type of switching module configured to selectively couple stimulation energy to selected electrodes 116, 118 and to selectively sense neurological brain signals with selected electrodes 116, 118. Hence, stimulation generator 202 can be coupled to electrodes 116, 118 via a switch module and conductors within leads 114. In some examples, however, IMD 106 does not include switch module 206.

Stimulation generator 202 may be a single channel or multi-channel stimulation generator. In particular, stimulation generator 202 may be capable of delivering a single stimulation pulse, multiple stimulation pulses, or a continuous signal at a given time via a single electrode combination or multiple stimulation pulses at a given time via multiple electrode combinations In some examples, each channel from stimulation generator 202 may be coupled to a respective electrode of electrodes 116, 118. In other examples, however, stimulation generator 202 and a switch module may be configured to deliver multiple channels on a time-interleaved basis. For example, a switch module may serve to time divide the output of stimulation generator 202 across different electrode combinations at different times to deliver multiple programs or channels of stimulation energy to patient 112.

Electrodes 116, 118 on respective leads 114 may be constructed of a variety of different designs. For example, one or both of leads 114 may include two or more electrodes at each longitudinal location along the length of the lead, such as multiple electrodes at different perimeter locations around the perimeter of the lead at each of the locations A, B, C, and D. On one example, the electrodes may be electrically coupled to a switch module via respective wires that are straight or coiled within the housing the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 114. These and other constructions may be used to create a lead with a complex electrode geometry.

Although sensing module 204 is incorporated into a common housing with stimulation generator 202 and processor 210 in FIG. 2, in other examples, sensing module 204 may be in a separate housing from IMD 106 and may communicate with processor 210 via wired or wireless communication techniques. Example neurological brain signals include, but are not limited to, a signal generated from local field potentials (LFPs) within one or more regions of brain 28. EEG and ECOG signals are examples of local field potentials that may be measured within brain 120. However, local field potentials may include a broader genus of electrical signals within brain 120 of patient 112. Instead of, or in addition to, LFPs, IMD 106 may be configured to detect patterns of single-unit activity and/or multi-unit activity. IMD 106 may sample this activity at rates above 1,000 Hz, and in some examples within a frequency range of 6,000 Hz to 40,000 Hz. IMD 106 may identify the wave-shape of single units and/or an envelope of unit modulation that may be features used to differentiate or rank electrodes. In some examples, this technique may include phase-amplitude coupling to the envelope or to specific frequency bands in the LFPs sensed from the same or different electrodes.

Sensor 212 may include one or more sensing elements that sense values of a respective patient parameter. For example, sensor 212 may include one or more accelerometers, optical sensors, chemical sensors, temperature sensors, pressure sensors, or any other types of sensors. Sensor 212 may output patient parameter values that may be used as feedback to control delivery of therapy. IMD 106 may include additional sensors within the housing of IMD 106 and/or coupled via one of leads 114 or other leads. In addition, IMD 106 may receive sensor signals wirelessly from remote sensors via telemetry module 208, for example. In some examples, one or more of these remote sensors may be external to patient (e.g., carried on the external surface of the skin, attached to clothing, or otherwise positioned external to the patient). For example, IMD 106 may determine from these one or more additional sensors the brain state of the patient and sense signals for determining electrode movement during a brain state of lower fluctuation or lower noise to improve signal detection. In other examples, IMD 106 may employ an inertial sensor to determine when the patient is at rest (e.g., lying down and/or sleeping) and sense signals for determining lead movement during a time of rest to reduce noise or other motion artifacts in the sensed signals. In some examples, IMD 106 may sense signals for determining lead movement in response to receiving an indication that the patient received a dose of medication or the patient has entered a physician appointment.

Telemetry module 208 supports wireless communication between IMD 106 and an external programmer 104 or another computing device under the control of processor 210. Processor 210 of IMD 106 may receive, as updates to programs, values for various stimulation parameters such as magnitude and electrode combination, from programmer 104 via telemetry module 208. The updates to the therapy programs may be stored within therapy programs 214 portion of memory 211. In addition, processor 210 may control telemetry module 208 to transmit alerts or other information to programmer 104 that indicate a lead moved with respect to tissue. Telemetry module 208 in IMD 106, as well as telemetry modules in other devices and systems described herein, such as programmer 104, may accomplish communication by radiofrequency (RF) communication techniques. In addition, telemetry module 208 may communicate with external medical device programmer 104 via proximal inductive interaction of IMD 106 with programmer 104. Accordingly, telemetry module 208 may send information to external programmer 104 on a continuous basis, at periodic intervals, or upon request from IMD 106 or programmer 104.

Power source 220 delivers operating power to various components of IMD 106. Power source 220 may include a small rechargeable or non-rechargeable battery and a power generation circuit to produce the operating power. Recharging may be accomplished through proximal inductive interaction between an external charger and an inductive charging coil within IMD 220. In some examples, power requirements may be small enough to allow IMD 220 to utilize patient motion and implement a kinetic energy-scavenging device to trickle charge a rechargeable battery. In other examples, traditional batteries may be used for a limited period of time.

According to the techniques of the disclosure, processor 210 of IMD 106 delivers, electrodes 116, 118 interposed along leads 114 (and optionally switch module 206), electrical stimulation therapy to patient 112. The DBS therapy is defined by one or more therapy programs 214 having one or more parameters stored within memory 211. For example, the one or more parameters include a current amplitude (for a current-controlled system) or a voltage amplitude (for a voltage-controlled system), a pulse rate or frequency, and a pulse width, or quantity of pulses per cycle. In examples where the electrical stimulation is delivered according to a “burst” of pulses, or a series of electrical pulses defined by an “on-time” and an “off-time,” the one or more parameters may further define one or more of a number of pulses per burst, an on-time, and an off-time.

In some examples, sensing module 204 may sense an electrical signal that is a neurological signal (e.g., a LFP signal) within the Beta frequency band of brain 120 of patient 112. The signal within the Beta frequency band of patient 112 may correlate to one or more symptoms of Parkinson's disease in patient 112. Generally speaking, neurological signals within the Beta frequency band of patient 112 may be approximately proportional to the severity of the symptoms of patient 112. For example, as tremor induced by Parkinson's disease increases, one or more of electrodes 116, 118 detect an increase in the magnitude of neurological signals within the Beta frequency band of patient 112. In this manner, the closest electrode combination to the origin of this neurological signal may be selected for therapy. When a lead rotates or shifts longitudinally, a different electrode combination may be best positioned to stimulate the tissue generating the neurological signal indicative of patient symptoms or of patient side-effects. Therefore, as described herein, processor 210 determines when this shift occurs with the electrodes and determines that the lead has moved. Processor 210 may automatically adjust the electrode combination for delivering therapy and/or other stimulation parameter values to compensate for the moved lead. Alternatively, processor 210 may transmit an alert to programmer 104 or other external device to indicate that updated stimulation parameters may be needed to continue efficacious therapy. For example, if the adjustments to electrode combinations and/or stimulation parameter values to compensate for the moved lead fall within respective ranges approved by the clinician, processor 210 may automatically adjust the electrode combination and/or other stimulation parameter values. If the adjustments to electrode combinations and/or stimulation parameter values to compensate for the moved lead do not fall within respective ranges approved by the clinician, processor 210 may communicate with programmer 104 to request approval or parameter values from a user.

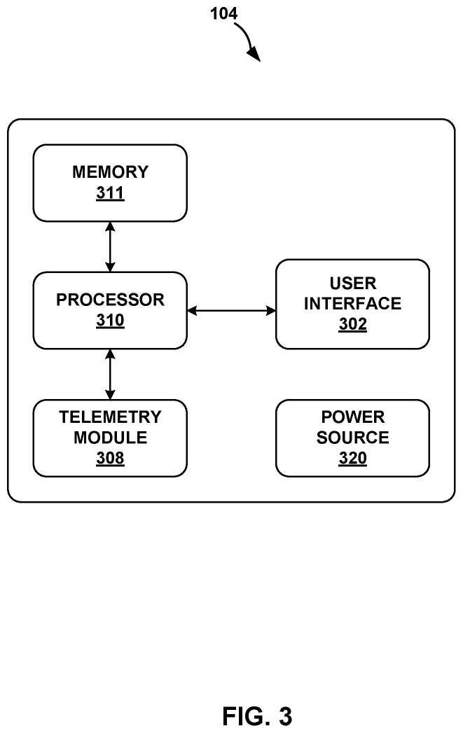

FIG. 3 is a block diagram of the external programmer 104 of FIG. 1 for controlling delivery of DBS therapy according to an example of the techniques of the disclosure. Although programmer 104 may generally be described as a hand-held device, programmer 104 may be a larger portable device or a more stationary device. In some examples, programmer 104 may be referred to as a tablet computing device. In addition, in other examples, programmer 104 may be included as part of a bed-side monitor, an external charging device or include the functionality of an external charging device. As illustrated in FIG. 3, programmer 104 may include a processor 310, memory 311, user interface 302, telemetry module 308, and power source 320. Memory 311 may store instructions that, when executed by processor 310, cause processor 310 and external programmer 104 to provide the functionality ascribed to external programmer 104 throughout this disclosure. Each of these components, or modules, may include electrical circuitry that is configured to perform some or all of the functionality described herein. For example, processor 310 may include processing circuitry configured to perform the processes discussed with respect to processor 310.

In general, programmer 104 comprises any suitable arrangement of hardware, alone or in combination with software and/or firmware, to perform the techniques attributed to programmer 104, and processor 310, user interface 302, and telemetry module 308 of programmer 104. In various examples, programmer 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. Programmer 104 also, in various examples, may include a memory 311, such as RAM, ROM, PROM, EPROM, EEPROM, flash memory, a hard disk, a CD-ROM, comprising executable instructions for causing the one or more processors to perform the actions attributed to them. Moreover, although processor 310 and telemetry module 308 are described as separate modules, in some examples, processor 310 and telemetry module 308 may be functionally integrated with one another. In some examples, processor 310 and telemetry module 308 correspond to individual hardware units, such as ASICs, DSPs, FPGAs, or other hardware units.

Memory 311 (e.g., a storage device) may store instructions that, when executed by processor 310, cause processor 310 and programmer 104 to provide the functionality ascribed to programmer 104 throughout this disclosure. For example, memory 311 may include instructions that cause processor 310 to obtain a parameter set from memory, select a spatial electrode movement pattern, provide an interface that recommends or otherwise facilitates parameter value selection, or receive a user input and send a corresponding command to IMD 106, or instructions for any other functionality. In addition, memory 311 may include a plurality of programs, where each program includes a parameter set that defines stimulation therapy.

User interface 302 may include a button or keypad, lights, a speaker for voice commands, a display, such as a liquid crystal (LCD), light-emitting diode (LED), or organic light-emitting diode (OLED). In some examples the display may be a touch screen. User interface 302 may be configured to display any information related to the delivery of stimulation therapy, identified patient behaviors, sensed patient parameter values, patient behavior criteria, or any other such information. User interface 302 may also receive user input via user interface 302. The input may be, for example, in the form of pressing a button on a keypad or selecting an icon from a touch screen.

Telemetry module 308 may support wireless communication between IMD 106 and programmer 104 under the control of processor 310. Telemetry module 308 may also be configured to communicate with another computing device via wireless communication techniques, or direct communication through a wired connection. In some examples, telemetry module 308 provides wireless communication via an RF or proximal inductive medium. In some examples, telemetry module 308 includes an antenna, which may take on a variety of forms, such as an internal or external antenna. In some examples, IMD 106 and/or programmer 104 may communicate with remote servers via one or more cloud-services in order to deliver and/or receive information between a clinic and/or programmer.

Examples of local wireless communication techniques that may be employed to facilitate communication between programmer 104 and IMD 106 include RF communication according to the 802.11 or Bluetooth specification sets or other standard or proprietary telemetry protocols. In this manner, other external devices may be capable of communicating with programmer 104 without needing to establish a secure wireless connection. As described herein, telemetry module 308 may be configured to transmit a spatial electrode movement pattern or other stimulation parameter values to IMD 106 for delivery of stimulation therapy.

According to the techniques of the disclosure, in some examples, processor 310 of external programmer 104 defines the parameters of a homeostatic therapeutic window, stored in memory 311, for delivering DBS to patient 112. In one example, processor 311 of external programmer 104, via telemetry module 308, issues commands to IMD 106 causing IMD 106 to deliver electrical stimulation therapy via electrodes 116, 118 via leads 114.

Many examples of electrode configurations for a medical lead are described herein. Specific examples are described in FIGS. 4A-17. Some of these examples may have similarities to other examples. In some situations, the same lead have an electrode configuration may be implanted within different hemispheres of the brain. In other examples, the leads in different hemispheres may have leads with different electrode configurations. For example, one lead for the left hemisphere may have a first asymmetric electrode configuration that is chiral to a second asymmetric electrode configuration for a different lead in the right hemisphere. In these multi-lead pairs, or sets, each lead may have electrodes at positions intended to deliver stimulation to, or sense signals from, a target tissue in each hemisphere of the brain.

In some examples, a medical lead includes a cylindrical lead body defining a longitudinal axis, a proximal portion, and a distal portion. The lead may include a first electrode including a first length parallel with the longitudinal axis, where the first electrode is disposed at a first circumferential position around the cylindrical lead body. The longitudinal axis may run through the center of the cylindrical body. In other examples of a non-cylindrical body, the axis may run through the center of the body. The lead may also include a plurality of second electrodes, each electrode of the plurality of second electrodes including one or more second lengths parallel with the longitudinal axis and different than the first length, where the plurality of second electrodes are disposed at a second circumferential position of the cylindrical lead body different than the first circumferential position. Each electrode of the plurality of second electrodes are disposed at different longitudinal positions along the length of the lead. In this manner, the lengths of each electrode may refer to the longitudinal length of each electrode that runs along the length of the lead. In contrast, the width of each electrode may be the dimension orthogonal to the longitudinal axis that follows the perimeter, or circumference, of the lead.

The lead may also include a third electrode at a third circumferential position different than the first circumferential position and the second circumferential position. In this manner, the first electrode, the plurality of second electrodes, and the third electrode may be disposed at different respective positions around the perimeter of the lead. In some examples, the third electrode has a third length equal to the first length. In some examples, the lead may also have a plurality of fourth electrodes at a fourth circumferential position different than the first circumferential position, the second circumferential position, and the third circumferential position. The first and third circumferential positions can be on opposing sides of the cylindrical lead body, and the second and fourth circumferential positions can be on opposing sides of the cylindrical lead body. The one or more second lengths of each second electrode of the plurality of second electrodes can be equal to one or more fourth lengths of each fourth electrode of the plurality of fourth electrodes. The one or more second lengths for at least two second electrodes of the plurality of second electrodes can be different, and the one or more fourth lengths of at least two fourth electrodes of the plurality of fourth electrodes can be different. In other words, the lengths of electrodes at the same circumferential position of the lead can be different from each other. In some examples, at least one second length of the one or more second lengths of the plurality of second electrodes is different from any of the one or more fourth lengths of the plurality of fourth electrodes.

In some examples, a plurality of third electrodes comprising a third length shorter than the second length of the second electrodes are disposed longitudinally between the plurality of second electrodes. In addition, or alternatively, the plurality of second electrodes and the plurality of third electrodes are disposed at the second circumferential position. In this way, multiple electrodes may be located at the same circumferential position and have different lengths from each other.

In some examples, a first circumferential width of the first electrode on a lead is smaller than a second circumferential width of each electrode of the plurality of the second electrodes of the lead. In this manner, the different electrodes may take up a different perimeter length from each other, or first electrode with the smaller circumferential width is “narrower” than the second electrodes. Some longer electrodes may be more narrow than, or have smaller widths in the circumferential direction than, shorter electrodes. In some examples, this difference in electrode width may be to reduce the surface area difference between the longer and shorter electrodes In some examples, the first surface area of the first electrode is approximately equal to a second surface area of each electrode of the plurality of second electrodes, even though the first electrode has a greater longitudinal length than the longitudinal length of the second electrodes.

In some examples, multiple shorter electrodes may cover a total longitudinal length equal to, or less than, the longitudinal length of a longer electrode at a different circumferential position. In one example, the different longitudinal positions of the plurality of second electrodes can be within a longitudinal distal end of the first electrode and a longitudinal proximal end of the first electrode (e.g., equal to, or less than the length of the first electrode). Even on a cylindrical lead where the electrodes are curved to correspond to the curve of the cylindrical lead body, the shape of the electrode may be referred to as a rectangular shape because of the opposing edges of the electrode are parallel with each other. In some examples, the corners of the electrodes are shape, but in other examples, the corners of the electrodes may be rounded corners.

Generally, the electrodes may be constructed of an electrically conductive material, such as a metal or metal alloy. In this manner, the electrodes may be more rigid than the cylindrical lead body carrying the electrodes. The medical lead can thus be configured to bend at locations proximal to and distal from the electrode instead of at the electrode. For longer electrodes that may cover a longer longitudinal length than other electrodes at a different circumferential position, the longer electrode may reduce or prevent bending between the shorter electrodes at the different circumferential position.

In some examples, the length of longer electrodes (e.g., longer vertical electrodes) may be selected from about 5 mm to about 20 mm. The longer electrodes may be shorter or longer than this range. In some examples, the longer electrodes may have a length equal to or greater than the length that multiple shorter electrodes cover at a different circumferential position. The length of shorter electrodes may be selected from about 1 mm to about 10 mm. However, the shorter electrodes may be shorter or longer than this range in other examples.

FIGS. 4A and 4B are conceptual diagrams of example leads 400 and 410, respectively, with respective electrodes carried by the lead. As shown in FIGS. 4A and 4B, leads 400 and 410 are embodiments of leads 114 shown in FIG. 1. As shown in FIG. 4A, lead 400 includes different electrodes have various lengths and mounted at different axial positions and different circumferential positions of lead housing 402. Lead 400 is inserted into through cranium 122 to a target position within brain 18.

Lead 400 is implanted within brain 120 at a location determined by the clinician to be near an anatomical region to be stimulated. Electrodes 404A and 404D are ring electrodes that each cover an entire circumference of lead housing 402. In between electrodes 404A and 404D are partial ring electrodes 404B and 404C located at different axial positions but the same circumferential position. Electrode 404E is longer than all of electrodes 404A-D and has a length equal to the length of lead housing 402 covered by electrodes 404B and 404C. Electrodes 404A, 404B, 404C, and 404D are equally spaced along the axial length of lead housing 402 at different axial positions. Electrodes of one circumferential location may be lined up on an axis parallel to the longitudinal axis of lead 400. Alternatively, electrodes of different axial positions may be staggered around the circumference of lead housing 402. In addition, lead 400 or 410 may include asymmetrical electrode locations around the circumference, or perimeter, of each lead or electrodes of the same level that have different sizes. These electrodes may include semi-circular electrodes that may or may not be circumferentially aligned between electrode levels.

Lead housing 402 may include a radiopaque stripe (not shown) along the outside of the lead housing. The radiopaque stripe corresponds to a certain circumferential location that allows lead 400 to the imaged when implanted in patient 112. Using the images of patient 112, the clinician can use the radiopaque stripe as a marker for the exact orientation of lead 400 within the brain of patient 112. Orientation of lead 400 may be needed to easily program the stimulation parameters by generating the correct electrode configuration to match the stimulation field defined by the clinician. In other embodiments, a marking mechanism other than a radiopaque stripe may be used to identify the orientation of lead 400. These marking mechanisms may include something similar to a tab, detent, or other structure on the outside of lead housing 402. In some embodiments, the clinician may note the position of markings along a lead wire during implantation to determine the orientation of lead 400 within patient 112. In some examples, programmer 104 may update the orientation of lead 400 in visualizations based on the movement of lead 400 from sensed signals.

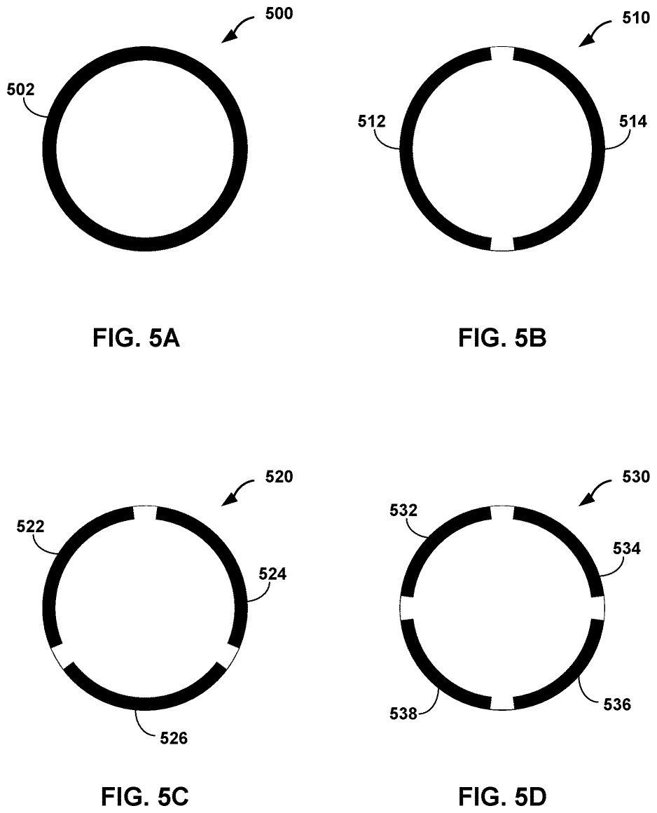

FIG. 4B illustrates lead 410 that includes multiple electrodes 414A-H at different respective circumferential positions at different axial and circumferential positions. Similar to lead 400, lead 410 is inserted through a burr hole in cranium 122 to a target location within brain 120. Lead 410 includes lead housing 412. Electrodes 414A-414H are located at the distal end of lead 410. Each electrode 414A-414H is evenly spaced from the adjacent electrode in the axial direction and only extends around a portion of the perimeter of lead body 412. The length of electrodes 414C and 414F are longer than the other electrodes, and may be equal to or different lengths from each other. However, electrodes 414C and 414F may only partially overlap each other in the axial direction. Two or more electrodes may be disposed around the perimeter of lead body 412 at any axial position, such as shown in FIGS. 5A-5D. Each electrode may be substantially rectangular in shape. Alternatively, the individual electrodes may have alternative shapes, e.g., circular, oval, triangular, rounded rectangles, or the like.

In alternative embodiments, electrodes 414A-414H may not be evenly spaced along the longitudinal axis of the respective leads 400 and 410. In some examples, any axial spaces between electrodes may be approximately 3 millimeters (mm), but the spacing may be less than 3 mm or greater than 3 mm such as up to 10 mm apart. Variable spaced electrodes in the axial direction may be useful in reaching target anatomical regions deep within brain 120 while avoiding potentially undesirable anatomical regions. Further, the electrodes in adjacent levels need not be aligned in the direction as the longitudinal axis of the lead, and instead may be oriented diagonally with respect to the longitudinal axis.

Leads 400 and 410 are substantially rigid to prevent the implanted lead from varying from the expected lead shape. Leads 400 or 410 may be substantially cylindrical in shape. In other embodiments, leads 400 or 410 may be shaped differently than a cylinder. For example, the leads may include one or more curves to reach target anatomical regions of brain 18. In some embodiments, leads 400 or 410 may be similar to a flat paddle lead or a conformable lead shaped for patient 12. Also, in other embodiments, leads 400 and 410 may any of a variety of different polygonal cross sections (e.g., triangle, square, rectangle, octagonal, etc.) taken transverse to the longitudinal axis of the lead.

FIGS. 5A-5D are transverse cross-sections of example stimulation leads having one or more electrodes around the circumference of the lead. As shown in FIGS. 5A-5D, one axial position along the length of leads 400 and 410 are illustrated to show electrode placement around the perimeter, or around the longitudinal axis, of the lead. FIG. 5A shows one circumferential electrode 502. Circumferential electrode 502 encircles the entire circumference of the lead perimeter and may be referred to as a ring electrode in some examples. Circumferential electrode 502 may be utilized as a cathode or anode as configured by the user interface.

FIG. 5B shows two electrodes 512 and 514. Each electrode 512 and 514 wraps approximately 170 degrees around the circumference of the lead. Spaces of approximately 10 degrees are located between electrodes 512 and 514 to prevent inadvertent coupling of electrical current between the electrodes. Smaller or larger spaces between electrodes (e.g., between 10 degrees and 30 degrees) may be provided in other examples. Each electrode 512 and 514 may be programmed to act as an anode or cathode.

FIG. 5C shows three equally sized electrodes 522, 524, and 526. Each electrode 522, 524, and 526 encompass approximately 110 degrees of the circumference of the lead. Similar to FIG. 5B, spaces of approximately 10 degrees separate electrodes 522, 524, and 526. Smaller or larger spaces between electrodes (e.g., between 10 degrees and 30 degrees) may be provided in other examples. Electrodes 522, 524, and 526 may be independently programmed as an anode or cathode for stimulation.

FIG. 5D shows four electrodes 532, 534, 536, and 538. Each electrode 532, 534, 536, and 538 covers approximately 80 degrees of the circumference with approximately 10 degrees of insulation space between adjacent electrodes. Smaller or larger spaces between electrodes (e.g., between 10 degrees and 30 degrees) may be provided in other examples. In other embodiments, up to ten or more electrodes may be included at the same axial position. In alternative embodiments, consecutive axial positions of lead 114 may include a variety of circumferential spacing of electrodes. For example, lead 114 (or any other lead described herein) may alternate between two electrodes at one axial position and four electrodes at another axial position. In this manner, various stimulation field shapes may be produced within brain 120 of patient 112. Further the above-described sizes of electrodes within an electrode level are merely examples, and the invention is not limited to the example electrode sizes.

Also, the insulation space, or non-electrode surface area, may be of any size. Generally, the insulation space is between approximately 1 degree and approximately 20 degrees. More specifically, the insulation space may be between approximately 5 and approximately 15 degrees. In other examples, insulation space may be between approximately 10 degrees and 30 degrees or larger. Smaller insulation spaces may allow a greater volume of tissue to be stimulated. In alternative embodiments, electrode size may be varied around the circumference of an electrode level. In addition, insulation spaces may vary in size as well. Such asymmetrical electrode levels may be used in leads implanted at tissues needing certain shaped stimulation fields. Although cylindrical leads are generally described herein, leads having a oval, rectangular, square, or asymmetrical cross-sectional shape may similarly have electrodes positioned at different locations around the perimeter of the lead as described herein with respect to cylindrical leads.

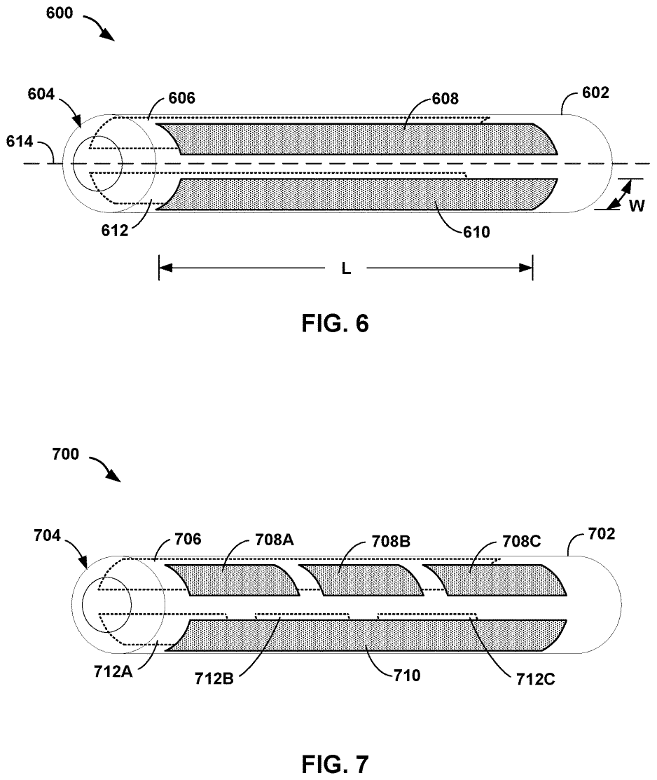

FIG. 6 is a perspective view of an example lead 600 with longitudinally orientated electrodes at respective circumferential positions. Lead 600 is an example of any of leads 114 or 138. As shown in the example of FIG. 6, lead 600 includes lead housing 602 that carries four electrodes 606, 608, 610, and 612 at different circumferential positions around the perimeter of lead housing 602. Electrodes 606 and 612 are shows as dotted lines because they are on the backside of lead housing 602 and obscured from view by lead housing 602. Distal end 604 may be a rounded end shape or any other shape, or, in some examples, include a tip electrode.

Each of electrodes 606, 608, 610, and 612 may be referred to as vertical electrodes because they all have a length “L” parallel with longitudinal axis 614 that is greater than the width “W” that runs in the direction of the perimeter of lead body 602. This manner, the width W is orthogonal to the length L for each electrode. In some examples, vertical electrodes may have a length to width ratio in a certain range of ratios. For example, vertical electrodes may have a length to width ratio from 1.2:1 to 20:1. In some examples, vertical electrodes may have a length to width ratio from 2:1 to 10:1. In some examples, vertical electrodes may have a length to width ratio from 2:1 to 5:1.

Electrodes 606, 608, 610, and 612 are shown has having the same longitudinal position, the same length, and the same width. In addition, electrodes 606, 608, 610, and 612 are shown to be spaced equally around the perimeter of lead body 602 (e.g., the spaces between electrodes are the same width). In other examples, one or more of electrodes 606, 608, 610, and 612 may have different longitudinal positions, different lengths, different widths, or different shapes from one or more other electrodes 606, 608, 610, and 612. In addition, or alternatively, the spaces between electrodes 606, 608, 610, and 612 may be different for one, some, or all of the spaces between 606, 608, 610, and 612 such that 606, 608, 610, and 612 are not evenly spaced around the perimeter of lead body 602.

In some examples, the length of longer vertical electrodes such as electrodes 606, 608, 610, and 612 may have a length in a range from 5 mm to 20 mm. In some examples, these longer vertical electrodes may be placed on a lead with other shorter vertical electrodes, such as some of the example leads of FIGS. 7-17. For example, these shorter vertical electrodes may have a length in a range from 1 mm to 10 mm. These lengths and widths described with respect to FIG. 6 may apply to any electrodes described herein, such as those for FIGS. 7-17 as well.

FIG. 7 is a perspective view of an example lead 700 with longitudinally orientated electrodes and longitudinal segmented electrodes at respective circumferential positions. Lead 700 is an example of any of leads 114 or 138 and may be similar to lead 600. As shown in the example of FIG. 7, lead 700 includes lead housing 702 that carries 8 electrodes 706, 708A-C, 710, and 712A-C at different circumferential positions around the perimeter of lead housing 702. Distal end 704 may be a rounded end shape or any other shape, or, in some examples, include a tip electrode.

Each of the electrodes may be referred to as vertical electrodes because they all have a length greater than the width. Longitudinal electrodes 706 and 710 may have the same length that is equal to the entire axial length of lead body 702 that is occupied by each set of electrodes 708A-C and 712A-C. Electrodes 708A-C and 712A-C may all have the same length that is shorter than the longer length of electrodes 706 and 710. Electrodes 706 and 710 may be disposed on opposing sides of lead housing 702, and electrodes 708A-C may be on the opposite side of lead housing 702 from electrodes 712A-C.

The different circumferential positions of electrode 706, 710, 708A-C, and 712A-C may be equally spaced around the perimeter of lead housing 702. The circumferential position of each electrode may refer to the centroid position of the electrode or the location of the width of the electrodes. Electrodes 708A-C are located at different axial positions along lead housing 702 and equally spaced. At the different circumferential position, electrodes 712A-C are located at different axial positions along lead housing 702 and equally spaced. Electrodes 708A and 712A may be located at a distal axial position, electrodes 708B and 712B may be located at a middle axial position, and 708C and 712C may be located at a proximal axial position. Although all of the electrodes of lead housing 702 are shown with the same width, the widths may be different in other examples. For example, the width of electrodes 706 and 710 may be smaller than the width of electrodes 708A-C and 712A-C. This smaller width may be selected to equalize the surface area of each of electrodes 706 and 710 to each of electrodes 708A-C and 712A-C. In some examples, the circumferential gaps between electrodes, such as between electrodes 708A and 10, may be equal to the longitudinal gaps between electrodes, such as between electrodes 708A and 708B. In other examples, the circumferential gaps may be different than longitudinal gaps.

As shown in the example of FIG. 7, electrode 706 may have a first length parallel with the longitudinal axis and disposed at a first circumferential position around the cylindrical lead body 702. A plurality of second electrodes 708A-C each have second lengths parallel with the longitudinal axis and different than the first length of electrode 706. Second electrodes 708A-C may be disposed at a second circumferential position of the cylindrical lead body 702 different than the first circumferential position, and each electrode of the plurality of second electrodes 708A-C are disposed at different longitudinal positions. Third electrode 710 is at a third circumferential position different than the first circumferential position and the second circumferential position, where the third electrode 710 has a third length equal to the first length. A plurality of fourth electrodes 712A-C are disposed at a fourth circumferential position different than the first circumferential position and the second circumferential position. The first and third circumferential positions of electrodes 706 and 710 are on opposing sides of the cylindrical lead body 702, and the second and fourth circumferential positions of electrodes 708A-C and 712A-C, respectively, are on opposing sides of the cylindrical lead body 702. The one or more second lengths of each second electrode of the plurality of second electrodes 708A-C are equal to one or more fourth lengths of each fourth electrode of the plurality of fourth electrodes 712-A-C. The different longitudinal positions of the plurality of second electrodes are within the longitudinal distal end of the first electrode and a longitudinal proximal end of the first electrode.

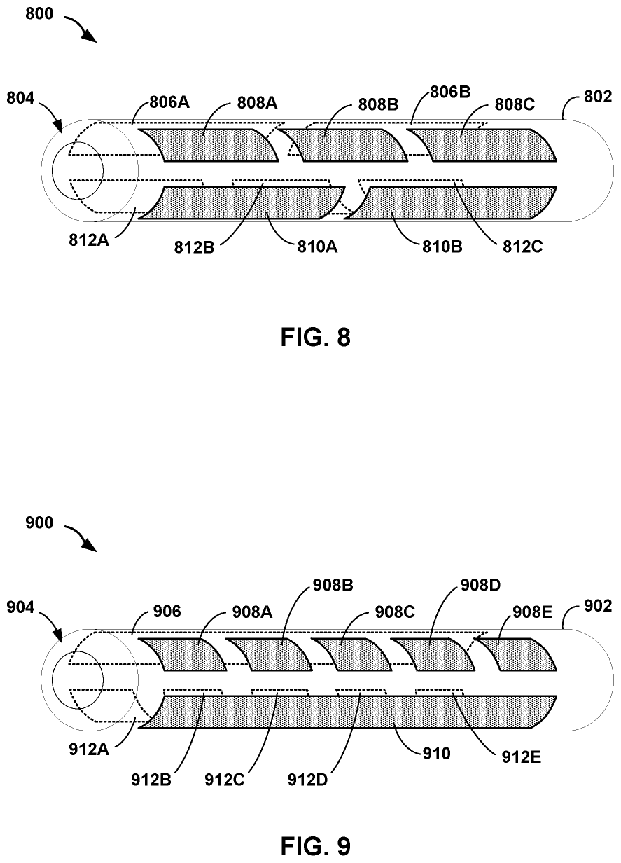

FIG. 8 is a perspective view of an example lead 800 with different longitudinal segmented electrodes at respective circumferential positions. Lead 800 is an example of any of leads 114 or 138 and may be substantially similar to lead 700 with the exception of the longest electrodes from lead 700 being segmented into two equal length electrodes. As shown in the example of FIG. 8, lead 800 includes lead housing 802 that carries 10 electrodes 806A-B, 808A-C, 810A-B, and 812A-C at different circumferential positions around the perimeter of lead housing 802. Distal end 804 may be a rounded end shape or any other shape, or, in some examples, include a tip electrode.

Electrodes 808A-C may be substantially similar to electrodes 708A-C of lead 700, and electrodes 808A-C may be substantially similar to electrodes 708A-C of lead 700. Electrodes 806A and 806B may be similar in length, and electrodes 812A and 812B may also be of similar length. Electrodes 806A-B may be positioned opposing electrodes 812A-B.

FIG. 9 is a perspective view of an example lead 900 with longitudinally orientated electrodes and longitudinal segmented electrodes at respective circumferential positions. Lead 900 is an example of any of leads 114 or 138 and may be substantially similar to lead 700 with the exception of the three shorter electrodes from lead 700 being segmented into smaller equal length electrodes. As shown in the example of FIG. 9, lead 900 includes lead housing 902 that carries 12 electrodes 906, 908A-E, 910, and 912A-E at different circumferential positions around the perimeter of lead housing 902. Distal end 904 may be a rounded end shape or any other shape, or, in some examples, include a tip electrode.

Electrode 906 may be substantially similar to electrode 706, and electrode 910 may be substantially similar to electrode 710 of lead 700. The five electrodes 908A-E may be similar in length to each other, and the five electrodes 912A-E may also be of similar length to each other. Electrodes 908A-E may be positioned opposing electrodes 912A-E. The longitudinal lengths of electrodes 908A-E and 912A-E may still be longer than the respective width of each electrode in some examples, but the length of electrodes 908A-E and 912A-E may be smaller than the width of the electrodes in other examples so that they are not vertical electrodes as described herein. In some examples, one or both of electrodes 906 or 910 may be segmented into two or more separate electrodes instead of the single long electrode.

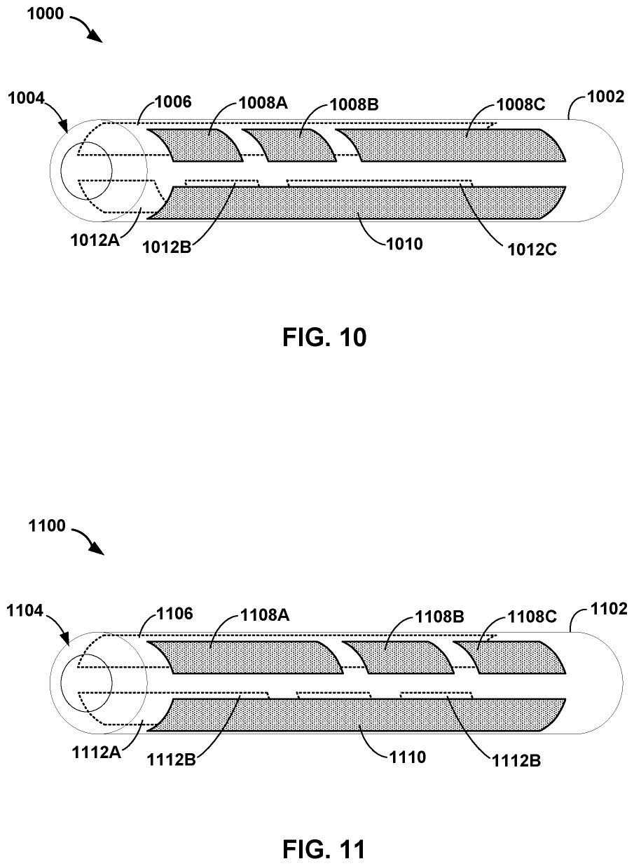

FIG. 10 is a perspective view of an example lead 1000 with longitudinally orientated electrodes and smaller longitudinal segmented electrodes at a distal end of the lead at respective circumferential positions. Lead 1000 is an example of any of leads 114 or 138 and may be substantially similar to lead 700 with the exception that the three shorter electrodes from lead 700 having unequal lengths to each other. As shown in the example of FIG. 10, lead 1000 includes lead housing 1002 that carries 8 electrodes 1006, 1008A-C, 1010, and 1012A-C at different circumferential positions around the perimeter of lead housing 1002. Distal end 1004 may be a rounded end shape or any other shape, or, in some examples, include a tip electrode.

Electrode 1006 may be substantially similar to electrode 706, and electrode 1010 may be substantially similar to electrode 710 of lead 700. The three electrodes 1008A-C may have different lengths from each other. For example, electrodes 1008A and 1008B may have the same lengths which are both smaller than the length of the longer electrode 1008C. Although the ratio of the length of electrode 1008C to the lengths of electrodes 1008A-B may be approximately 3:1 in the example of FIG. 10, the ratio may be in a range of 1.1:1 to 10:1 in some examples. The spacing between electrodes 1008A-C may be equal or unequal. Electrodes 1008A and 1008B are disposed on the distal end of lead body 1002, close to distal end 1004. Electrodes 1012A-C may be similar to, and mirror electrodes 1008A-C. For example, electrodes 1012A and 1012B are also disposed near distal end 1004 and distal from electrode 1012C. In some examples, electrodes 1008A-B, and/or 1012A-B, may have different lengths from each other. In some examples, one or both of electrodes 1006 or 1010 may be segmented into two or more separate electrodes instead of the single long electrode.

FIG. 11 is a perspective view of an example lead 1000 with longitudinally orientated electrodes and smaller longitudinal segmented electrodes disposed proximal from the end of the lead and at respective circumferential positions. Lead 1100 is an example of any of leads 114 or 138 and may be substantially similar to lead 1000 of FIG. 10. However, lead 1100 includes smaller electrodes closer to the proximal end of the electrode portion of lead body 1102.

The three electrodes 1108A-C may have different lengths from each other. For example, electrodes 1108A and 1108B may have the same lengths which are both smaller than the length of the longer electrode 1108C. Although the ratio of the length of electrode 1108C to the lengths of electrodes 1108A-B may be approximately 3:1 in the example of FIG. 11, the ratio may be in a range of 1.1:1 to 10:1 in some examples. The spacing between electrodes 1108A-C may be equal or unequal. Electrodes 1108B and 1108C are disposed proximal of electrode 1108A such that electrode 1108A is closest to distal end 1104 of lead body 1102. Electrodes 1112A-C may be similar to, and mirror electrodes 1108A-C. For example, electrodes 1112B and 1112C are also disposed further from distal end 1104 than electrode 1112A. In some examples, electrodes 1108B-C, and/or 1112B-C, may have different lengths from each other.

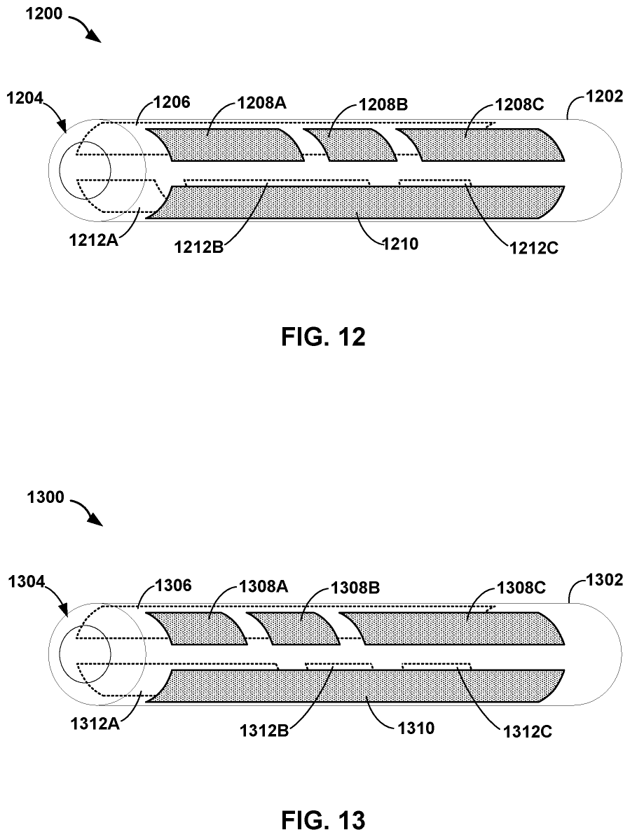

FIG. 12 is a perspective view of an example lead 1200 with longitudinally orientated electrodes and smaller longitudinal segmented electrodes disposed between the longer longitudinally orientated electrodes. Lead 1200 is an example of any of leads 114 or 138 and may be substantially similar to lead 1000 of FIG. 10. However, lead 1200 includes smaller electrodes at different longitudinal positions at different circumferential positions.

The three electrodes 1208A-C may have different lengths from each other. For example, electrodes 1208A and 1208C may have the same lengths which are both longer than the length of the shorter electrode 1208B. Although the ratio of the length of electrodes 1208A and 1208C to the lengths of electrodes 1208C may be approximately 3:1 in the example of FIG. 12, the ratio may be in a range of 1.1:1 to 10:1 in some examples. The spacing between electrodes 1208A-C may be equal or unequal. Electrodes 1208A and 1108C are disposed proximal and distal, respectively, of the middle and shorter electrode 1208B. Electrodes 1212A-C may be similar to electrodes 1208A-C, but length of the electrodes are switched such that the middle electrode 1208B is longer than both of electrodes 1212A and 1212C. In some examples, electrodes 1208A and C, and/or 1212A and C, may have different lengths from each other.

FIG. 13 is a perspective view of an example lead 1300 with longitudinally orientated electrodes and smaller longitudinal segmented electrodes disposed proximal from the end of the lead at one circumferential position and smaller longitudinal segmented electrodes disposed distal from the end of the lead at a different circumferential position. Lead 1300 is an example of any of leads 114 or 138 and may be substantially similar to lead 1000 of FIG. 10. However, lead 1300 includes smaller electrodes closer to the proximal end on one side of the lead body 1302 and smaller electrodes closer to the distal end 1304 on the other side of the lead body 1302.

The three electrodes 1308A-C may have different lengths from each other. For example, electrodes 1308A and 1308B may have the same lengths which are both smaller than the length of the longer electrode 1308C. Although the ratio of the length of electrode 1308C to the lengths of electrodes 1308A-B may be approximately 3:1 in the example of FIG. 13, the ratio may be in a range of 1.1:1 to 10:1 in some examples. The spacing between electrodes 1308A-C may be equal or unequal. Electrodes 1308A and 1308B are disposed distal of electrode 1308C such that electrode 1308A is closest to distal end 1304 of lead body 1302. Electrodes 1312A-C may be similar to, but flip the longitudinal locations of electrodes 1308A-C. For example, electrodes 1312B and 1312C are also disposed further from distal end 1104 than electrode 1312A.

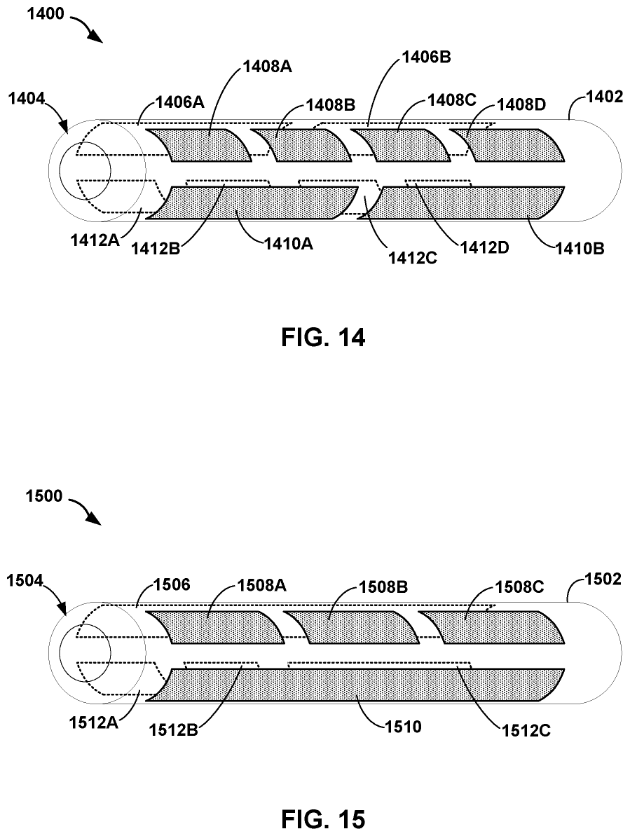

FIG. 14 is a perspective view of an example lead 1400 with different sized segmented electrodes at different longitudinal and circumferential positions. Lead 1400 is an example of any of leads 114 or 138 and may be substantially similar to lead 800 with the exception of shorter electrodes from lead 800 being segmented into four equal length electrodes instead of three electrodes. As shown in the example of FIG. 14, lead 1400 includes lead housing 1402 that carries 12 electrodes 1406A-B, 1408A-D, 1410A-B, and 1412A-D at different circumferential positions around the perimeter of lead housing 1402. Distal end 1404 may be a rounded end shape or any other shape, or, in some examples, include a tip electrode.

Electrodes 1408A-D may include four equal length and equally spaced electrodes. Electrodes 14012A-D may be substantially similar to, and on the opposing side of lead body 1402 from, electrodes 1408A-D. In other examples, one or more of electrodes 1408A-D and/or 1412A-D may have different lengths from other electrodes at the same circumferential position. Electrodes 1406A and 1406B may be similar in length, and electrodes 1412A and 1412B may also be of similar length. Electrodes 1406A-B may be positioned opposing electrodes 1412A-B.

FIGS. 15-17 are perspective views of an example leads with longitudinally orientated electrodes and different sized segmented electrodes at different longitudinal and circumferential positions. In the example of FIG. 15, lead 1500 is an example of any of leads 114 or 138 and may be substantially similar to lead 1000 of FIG. 10. However, lead 1500 includes different length shorter electrodes on one side of lead body 1502 from the lengths of the shorter electrodes on other side of lead 1502.

The three electrodes 1508A-C may have equal lengths from each other. The spacing between electrodes 1508A-C may be equal or unequal. On the opposing side of lead body 1502, electrodes 1512A-C include two shorter electrodes 1512A and 1512B located distal from the longer electrode 1512C (similar to electrodes 1012A-C of lead 1000). In this manner, the lengths of multiple electrodes (1508A-C) at one circumferential position of lead body 1502 may be different from the length of any of multiple electrodes (1512A-C) at the different circumferential position of lead body 1502.

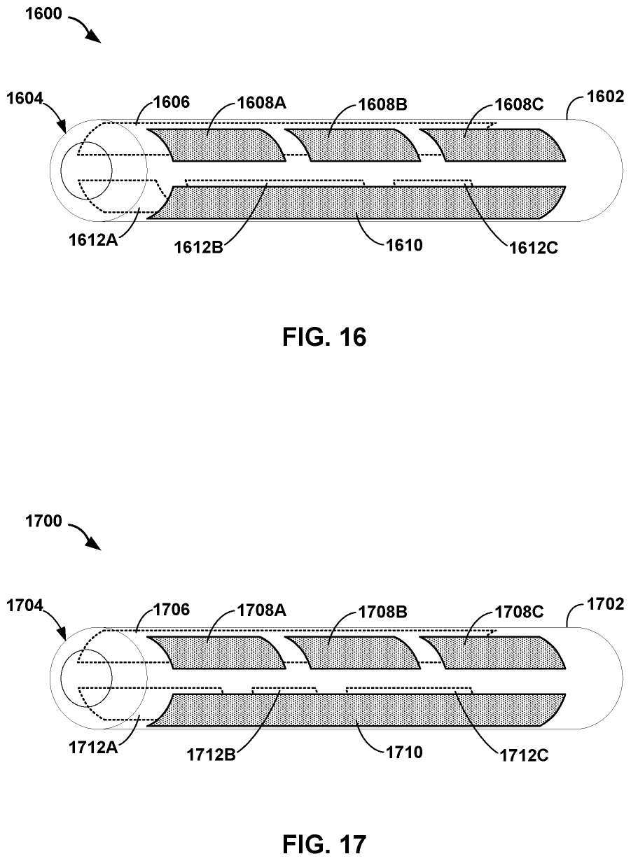

In the example of FIG. 16, lead 1600 is an example of any of leads 114 or 138 and may be substantially similar to lead 1500 of FIG. 15. However, lead 1600 includes different locations of shorter electrodes on one side of lead body 1602. The three electrodes 1608A-C may have equal lengths from each other. The spacing between electrodes 1608A-C may be equal or unequal. On the opposing side of lead body 1602, electrodes 1612A-C include two shorter electrodes 1612A and 1612C located distal and proximal from middle longer electrode 1612B, respectively. Electrodes 1612A-C may be similar to electrodes 1212A-C of lead 1200. In this example, the lengths of multiple electrodes (1608A-C) at one circumferential position of lead body 1602 may be different from the length of any of multiple electrodes (1612A-C) at the different circumferential position of lead body 1602.

In the example of FIG. 17, lead 1700 is an example of any of leads 114 or 138 and may be substantially similar to lead 1600 of FIG. 16. However, lead 1700 includes different locations of shorter electrodes on one side of lead body 1702. The three electrodes 1708A-C may have equal lengths from each other. The spacing between electrodes 1708A-C may be equal or unequal. On the opposing side of lead body 1702, electrodes 1712A-C include two longer electrodes 1712A and 1712C located distal and proximal from middle shorter electrode 1712B, respectively. Electrodes 1712A-C may be similar to electrodes 1208A-C of lead 1200. In this example, the lengths of multiple electrodes (1708A-C) at one circumferential position of lead body 1702 may be different from the length of any of multiple electrodes (1712A-C) at the different circumferential position of lead body 1702.

The leads in the examples if FIGS. 6-17 include electrodes at four different circumferential positions. However, in other examples, any lead may include two different circumferential positions, three different circumferential positions, or more than four different circumferential positions for the different electrodes. The electrodes are different circumferential positions may be equally spaced around the perimeter of the lead or spaced unequally from each other. In general, the electrodes at one circumferential position are separated from electrodes at another circumferential position by a continuous longitudinal space. However, in other examples, electrodes at different longitudinal positions may be staggered and partially overlapping electrodes at other longitudinal positions. In addition, at each circumferential position, the lead may include any number of electrodes along the longitudinal direction of the lead.

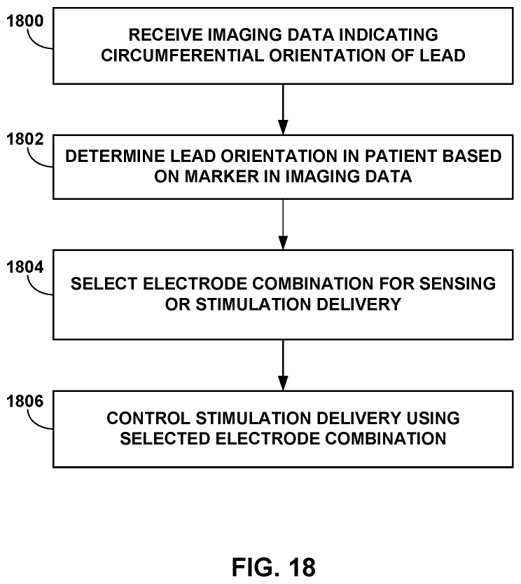

FIG. 18 is a flowchart illustrating an example technique for selecting electrodes for stimulation. The technique of FIG. 18 will be described with respect to processor 310 of IMD 106 in FIG. 3 and lead 700 of FIG. 7. However, other processors, devices, or combinations thereof, may perform the techniques of FIG. 18 in other examples to identify electrode position, select electrode combinations, and deliver stimulation.

As shown in FIG. 18, processor 310 receives initial imaging data indicating the circumferential orientation of lead 700 with respect to patient anatomy (1800). For example, the imaging data may be an MRI image, a CT image, or x-ray image in which one or more markers (e.g., radiopaque markers) on lead 700 can be detected. In some examples, user input may be provided to programmer 104 in order for processor 310 to have additional data related to lead orientation. From this imaging data, processor 310 may determine the lead orientation of lead 700 within the patient based on the one or more markers in the imaging data (1802). In some examples, the marker may include one or two triangular markers located at different circumferential positions of lead 700. In other examples where the electrode configuration on the lead is not symmetrical, such as leads 400, 410, 1200, 1300, 1500, 1600, or 1700, processor 310 may use the actual location of identified electrodes in the imaging data to determine the orientation of the lead instead of separate markers.

After the lead orientation is determined, processor 310 may select an electrode combination from the electrodes of lead 700 for sensing signals and/or delivering stimulation (1804). Processor 310 can then control stimulation delivery and/or sensing of electric signals using the electrode combination that was selected (1806). For example, processor 310 may control programmer 104 to transmit the electrode combination to IMD 106 for stimulation and/or sensing.

One or more examples are described herein.

Example 1. A medical lead comprising: a cylindrical lead body defining a longitudinal axis, a proximal portion, and a distal portion; a first electrode comprising a first length parallel with the longitudinal axis, the first electrode disposed at a first circumferential position around the cylindrical lead body; and a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions.

Example 2. The medical lead of example 1, further comprising a third electrode at a third circumferential position different than the first circumferential position and the second circumferential position, wherein the third electrode has a third length equal to the first length.

Example 3. The medical lead of example 2, further comprising a plurality of fourth electrodes at a fourth circumferential position different than the first circumferential position and the second circumferential position.

Example 4. The medical lead of example 3, wherein the first and third circumferential positions are on opposing sides of the cylindrical lead body, and wherein the second and fourth circumferential positions are on opposing sides of the cylindrical lead body.

Example 5. The medical lead of any of examples 3 and 4, wherein the one or more second lengths of each second electrode of the plurality of second electrodes are equal to one or more fourth lengths of each fourth electrode of the plurality of fourth electrodes.

Example 6. The medical lead of any of examples 3 and 4, wherein the one or more second lengths for at least two second electrodes of the plurality of second electrodes are different, and wherein one or more fourth lengths of at least two fourth electrodes of the plurality of fourth electrodes are different.

Example 7. The medical lead of example 6, wherein at least one second length of the one or more second lengths of the plurality of second electrodes is different from any of the one or more fourth lengths of the plurality of fourth electrodes.

Example 8. The medical lead of any of examples 1 through 7, wherein a first circumferential width of the first electrode is smaller than a second circumferential width of each electrode of the plurality of the second electrodes.

Example 9. The medical lead of any of examples 1 through 8, wherein a first surface area of the first electrode is approximately equal to a second surface area of each electrode of the plurality of second electrodes.

Example 10. The medical lead of any of examples 1 through 9, wherein the different longitudinal positions of the plurality of second electrodes are within a longitudinal distal end of the first electrode and a longitudinal proximal end of the first electrode.

Example 11. The medical lead of any of examples 1 through 10, wherein the first electrode and each electrode of the plurality of second electrodes comprise a rectangular shape having rounded corners.

Example 12. The medical lead of any of examples 1 through 11, wherein the first electrode is more rigid than the cylindrical lead body such that the medical lead to configured bend at locations proximal to and distal from the first electrode instead of at the first electrode.

Example 13. The medical lead of any of examples 1 through 12, wherein a plurality of third electrodes comprising a third length shorter than the second length of the second electrodes are disposed longitudinally between the plurality of second electrodes.

Example 14. The medical lead of example 13, wherein the plurality of second electrodes and the plurality of third electrodes are disposed at the second circumferential position.

Example 15. The medical lead of any of examples 1 through 14, wherein the first length is from 5 mm to 20 mm.

Example 16. The medical lead of any of examples 1 through 15, wherein the second length is from 1 mm to 10 mm.

Example 17. A system comprising: a medical lead comprising: a cylindrical lead body defining a longitudinal axis, a proximal portion, and a distal portion; a first electrode comprising a first length parallel with the longitudinal axis, the first electrode disposed at a first circumferential position around the cylindrical lead body; a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions; a plurality of contacts disposed on the proximal portion of the cylindrical lead; and a plurality of conductors electrically coupling the first electrode and the plurality of second electrodes to respective contacts of the plurality of contacts; and an implantable medical device configured to: electrically couple to the plurality of contacts of the medical lead; and deliver electrical stimulation via at least two electrodes of the first electrode and the plurality of second electrodes of the medical lead.

Example 18. The system of example 17, wherein the medical lead further comprises: a third electrode at a third circumferential position different than the first circumferential position and the second circumferential position, wherein the third electrode has a third length equal to the first length; and a plurality of fourth electrodes at a fourth circumferential position different than the first circumferential position, the second circumferential position, and the third circumferential position.

Example 19. The system of any of examples 17 and 18, wherein the implantable medical device comprises processing circuitry and stimulation generation circuitry, and wherein the processing circuitry is configured to select an electrode combination of any electrodes carried by the medical lead for delivering the electrical stimulation.

Example 20. A medical lead comprising: a cylindrical lead body defining a longitudinal axis, a proximal portion, and a distal portion; a first electrode comprising a first length parallel with the longitudinal axis, the first electrode disposed at a first circumferential position around the cylindrical lead body; a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions; a third electrode at a third circumferential position different than the first circumferential position and the second circumferential position, wherein the third electrode has a third length equal to the first length; and a plurality of fourth electrodes at a fourth circumferential position different than the first circumferential position and the second circumferential position, wherein the first and third circumferential positions are on opposing sides of the cylindrical lead body, and wherein the second and fourth circumferential positions are on opposing sides of the cylindrical lead body.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A medical lead comprising:
a cylindrical lead body defining a longitudinal axis, a proximal portion, and a distal portion;
a first electrode comprising a first length parallel with the longitudinal axis, the first electrode disposed at a first circumferential position around the cylindrical lead body; and
a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions.

2. The medical lead of claim 1, further comprising a third electrode at a third circumferential position different than the first circumferential position and the second circumferential position, wherein the third electrode has a third length equal to the first length.

3. The medical lead of claim 2, further comprising a plurality of fourth electrodes at a fourth circumferential position different than the first circumferential position and the second circumferential position.

4. The medical lead of claim 3, wherein the first and third circumferential positions are on opposing sides of the cylindrical lead body, and wherein the second and fourth circumferential positions are on opposing sides of the cylindrical lead body.

5. The medical lead of claim 3, wherein the one or more second lengths of each second electrode of the plurality of second electrodes are equal to one or more fourth lengths of each fourth electrode of the plurality of fourth electrodes.

6. The medical lead of claim 3, wherein the one or more second lengths for at least two second electrodes of the plurality of second electrodes are different, and wherein one or more fourth lengths of at least two fourth electrodes of the plurality of fourth electrodes are different.

7. The medical lead of claim 6, wherein at least one second length of the one or more second lengths of the plurality of second electrodes is different from any of the one or more fourth lengths of the plurality of fourth electrodes.

8. The medical lead of claim 1, wherein a first circumferential width of the first electrode is smaller than a second circumferential width of each electrode of the plurality of the second electrodes.

9. The medical lead of claim 1, wherein a first surface area of the first electrode is approximately equal to a second surface area of each electrode of the plurality of second electrodes.

10. The medical lead of claim 1, wherein the different longitudinal positions of the plurality of second electrodes are within a longitudinal distal end of the first electrode and a longitudinal proximal end of the first electrode.

11. The medical lead of claim 1, wherein the first electrode and each electrode of the plurality of second electrodes comprise a rectangular shape having rounded corners.

12. The medical lead of claim 1, wherein the first electrode is more rigid than the cylindrical lead body such that the medical lead to configured bend at locations proximal to and distal from the first electrode instead of at the first electrode.

13. The medical lead of claim 1, wherein a plurality of third electrodes comprising a third length shorter than the second length of the second electrodes are disposed longitudinally between the plurality of second electrodes.

14. The medical lead of claim 13, wherein the plurality of second electrodes and the plurality of third electrodes are disposed at the second circumferential position.

15. (canceled)

16. The medical lead of claim 1, wherein the first length is from 5 mm to 20 mm.

17. The medical lead of claim 1, wherein the second length is from 1 mm to 10 mm.

18. A system comprising:
a medical lead comprising:
a cylindrical lead body defining a longitudinal axis, a proximal portion, and a distal portion;
a first electrode comprising a first length parallel with the longitudinal axis, the first electrode disposed at a first circumferential position around the cylindrical lead body;
a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions;
a plurality of contacts disposed on the proximal portion of the cylindrical lead; and
a plurality of conductors electrically coupling the first electrode and the plurality of second electrodes to respective contacts of the plurality of contacts; and
an implantable medical device configured to:
electrically couple to the plurality of contacts of the medical lead; and
deliver electrical stimulation via at least two electrodes of the first electrode and the plurality of second electrodes of the medical lead.

19. The system of claim 18, wherein the medical lead further comprises:
a third electrode at a third circumferential position different than the first circumferential position and the second circumferential position, wherein the third electrode has a third length equal to the first length; and
a plurality of fourth electrodes at a fourth circumferential position different than the first circumferential position, the second circumferential position, and the third circumferential position.

20. The system of any of claim 18, wherein the implantable medical device comprises processing circuitry and stimulation generation circuitry, and wherein the processing circuitry is configured to select an electrode combination of any electrodes carried by the medical lead for delivering the electrical stimulation.

21. A medical lead comprising:
a cylindrical lead body defining a longitudinal axis, a proximal portion, and a distal portion;
a first electrode comprising a first length parallel with the longitudinal axis, the first electrode disposed at a first circumferential position around the cylindrical lead body;
a plurality of second electrodes, each electrode of the plurality of second electrodes comprising one or more second lengths parallel with the longitudinal axis and different than the first length, the plurality of second electrodes disposed at a second circumferential position of the cylindrical lead body different than the first circumferential position, and wherein each electrode of the plurality of second electrodes are disposed at different longitudinal positions;
a third electrode at a third circumferential position different than the first circumferential position and the second circumferential position, wherein the third electrode has a third length equal to the first length; and
a plurality of fourth electrodes at a fourth circumferential position different than the first circumferential position and the second circumferential position, wherein the first and third circumferential positions are on opposing sides of the cylindrical lead body, and wherein the second and fourth circumferential positions are on opposing sides of the cylindrical lead body.
